(19) United States
(12) Patent Application Publication
(10) Pub. No.: US 2021/0350885 A1
(43) Pub. Date: Nov. 11, 2021

(54) METHOD AND SYSTEM FOR INTERACTIVE PERSONAL WELLNESS RECOMMENDATIONS

(71) Applicant: gWell, Inc., Lexington, MA (US)

(72) Inventors: Surya Singh, Lexington, MA (US); Chester M. Hedgepeth, III, Westwood, MA (US)

(21) Appl. No.: 17/313,879

(22) Filed: May 6, 2021

Related U.S. Application Data
(60) Provisional application No. 63/022,183, filed on May 8, 2020.

(51) Int. Cl. G16H 10/60; A61B 5/00; A61B 5/15; G16H 10/20; G16H 20/00; G16H 50/20; G16H 50/30

(52) U.S. Cl. CPC G16H 10/60; A61B 5/6802; A61B 5/150824; A61B 2560/02; G16H 20/00; G16H 50/20; G16H 50/30; G16H 10/20

(57) ABSTRACT

A method and system for providing interactive wellness recommendations is disclosed. The system stores information regarding health profiles, each profile comprised of health data. An individual user health profile can be associated with at least one cohort group. The system obtains individual user health data to develop a user health profile and assigns the user to at least one cohort group based on the user health profile. The system also obtains a user wellness goal. Using the user health profile, a recommendation engine generates a user wellness recommendation for obtaining the user wellness goal based on the user's membership in at least one cohort group by correlating the particular health profile against effective wellness regimens for the user wellness goal as it relates to the cohort group. The user wellness recommendation may then be provided to the user. The user may be continually monitored through any number of methods. As new user health data is obtained, the system can monitor user progress towards their wellness goal and revise the user wellness recommendation based on that progress.

### RELATED APPLICATION

This application claims the benefit of U.S. Provisional Application No. 63/022,183, filed on May 8, 2020. The entire teachings of the above application are incorporated herein by reference.

### BACKGROUND

Across the world, people are increasingly focused on maintaining their health and wellness. Personal activity monitors can count steps taken, measure heart rate, provide estimates of calories burned, measure sleep quality, and compute calorie intake. Some may display a user's progress in real time, or may show it later on a smart phone, tablet, or computer. Some activity monitors can also provide users with some insight about their habits and health. In addition, the development of wearable sensors has allowed users and their healthcare professionals to monitor and detect abnormal physiological parameters. This technology has transformed healthcare by allowing continuous monitoring of patients without hospitalization.

In addition, modern advances in personalized medicine and wellness have allowed health care providers to take advantage of technology that can analyze a patient's fundamental biology, DNA, RNA, or protein, which ultimately leads to confirmation of particular conditions or disease. For example, personalized techniques such as genome sequencing can reveal mutations in DNA that influence diseases such as cystic fibrosis, or cancer.

### SUMMARY

Most health systems are constructed to provide the most sophisticated care to the sick. Some of these sophisticated systems employ diagnostic testing for selecting appropriate and optimal therapies based on the context of a patient's genetic content or other molecular or cellular analysis. The use of genetic information can play a major role in certain aspects of personalized medicine. On the other hand, existing systems that help individuals maximize their health and wellness before they become sick are typically less sophisticated. Direct to consumer products and services for promoting health and wellness are rapidly proliferating. These systems that assist individuals with their health and wellness tend to be generic products designed to help track very basic user wellness activity such as physical activity or changes in weight, and are relatively unsophisticated.

Embodiments consistent with principles of the present invention provide a method and system for providing interactive wellness recommendations. The system stores information regarding health profiles, each profile comprised of health data including a variety of health and wellness characteristics. Health data refers generally to a current state of being relating to physical and mental conditions. For example, the health data captured may include the existence or absences of particular diseases or conditions, or information concerning injury. Such information may be obtained from diagnostic testing, or from a health care professional. In some embodiments, the health data may also include genetic information, such as particular genetic panels linked with certain conditions or diseases. Multiple genes collectively influence the likelihood of developing a variety of conditions or diseases, and knowledge of these gene combinations may be used to predict a person's risk for a particular disease. Such information may be obtained by genotyping, or obtaining an individual's DNA sequence by using biological assays.

Cohort groups may be created based on various characteristics such as age, race, gender, height, weight, body fat percentage, activity level, or genotypical information. Further, the cohort groups may be organized by individual characteristics or groups of characteristics (e.g. men between the age of 30 to 40). An individual user health profile can be associated with at least one cohort group.

The system obtains individual user health data to develop a user health profile and assigns the user to at least one cohort group based on the user health profile. The system also obtains a user wellness goal, either directly from the user or from a health care professional that may be working with the user. The system may then access the user health profile and user wellness goal to generate a user wellness recommendation for a user wellness goal, at a recommendation engine, based on user profile's membership in at least one cohort group. The recommendation engine may generate the wellness recommendation by correlating the particular health profile against effective wellness regimens for the user wellness goal as it relates to the cohort group. Wellness refers to quality or state of being in good health, and wellness recommendations may include the practice of habits or choices that may lead to better health outcomes. In some embodiments, the recommendation engine may look at multiple cohort groups that the profile belongs to in order to determine a wellness recommendation among a selection of different recommendations. Once this user wellness recommendation information has been generated, it can be provided to the user and/or their health professional through some interface, such as a display. The user may be continually monitored through any number of methods, including through personal health monitoring devices. As new user health data is obtained, the system can analyze user compliance with the wellness recommendation and the monitored user health data to determine the user's progress towards their wellness goal. The system can then revise the user wellness recommendation based on that progress.

In some embodiments consistent with principles of the invention, user wellness goals may include pain management. In some of these embodiments, the health profile data may include genetic markers related to orthopedic conditions. Orthopedic conditions are injuries and diseases that affect the musculoskeletal system. This system includes the muscles, bones, nerves, joints, ligaments, tendons, and other connective tissues. The recommendation engine may compare the user heath profile to health intelligence data to generate the user wellness recommendation. Health intelligence data may be information provided by health care professionals relating to treatments or observations concerning various cohort groups. Wellness recommendations may be derived by correlating effective pain management outcomes among a user's cohort group.

In other embodiments consistent with principles of the invention, the system may monitor user health data through the use of survey data from the user. Such data may be obtained through a user interface of a software application. Similarly, the user wellness recommendation may be provided through the interface, or sent to the user through an e-mail or text message.

In other embodiments consistent with principles of the invention, the system may obtain progress data relating to outcomes associated with compliance with the user wellness recommendation and revise health data associated with at least one cohort group to which the user is assigned. Some embodiments may further include a progress engine to access the user wellness recommendation and periodic monitored user wellness data to generate wellness tracking data and provide the wellness tracking data through a display device.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows a system for that may be used to provide interactive personal wellness recommendations according to principles of the invention.

FIG. 2 is a flow diagram of various steps of a method for providing interactive personal wellness recommendations according to principles of the present invention.

FIG. 3 is a schematic diagram of the data flow according to principles of the present invention.

FIG. 4 is a representation of the data stored in connection with a health profile used in systems according to principles of the present invention.

FIGS. 5A-5L are example interfaces of a mobile application that may be used in connection with principles of the present invention.

### DETAILED DESCRIPTION

A description of example embodiments follows.

Embodiments described in the present disclosure provide systems, apparatus, and computer implemented methods for providing interactive personal wellness recommendations. The wellness recommendations may be provided according to a user's wellness goals, as well as a user's particular health profile, including genomic based information. This user health profile data may provide a way of assigning the user to at least one cohort group that may have some wellness data associated it. By correlating the user's particular health profile against the effective wellness regimens or other wellness information from the various cohort groups, recommendations may be provided to a user.

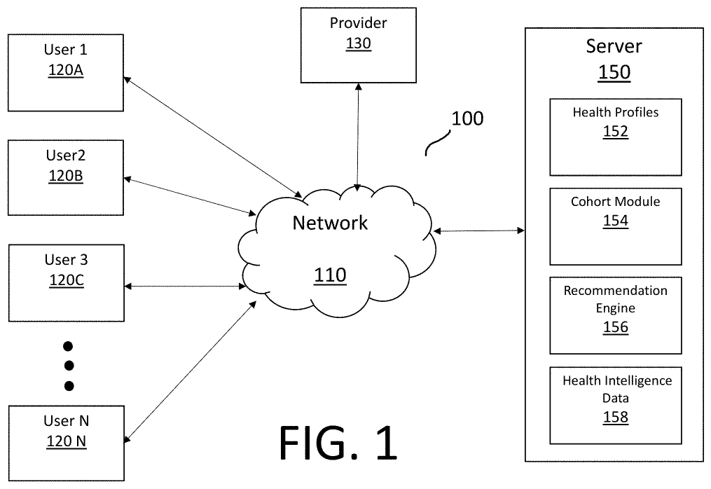

FIG. 1. illustrates a system 100 that may be used to provide interactive personal wellness recommendations according to principles of the invention. A user may access the system through an application or a web page on client device 120A that is connected to a server 150 through a network 110. Other users may access the server through individual client devices 120B-120N. The client devices 120A-120N may be handheld mobile devices, tablets, personal computers, or other means of interfacing a network. Generally, the network 110 may be the Internet, or in some embodiments a local area network, or in other embodiments a combination of the two. Within the server 150, information regarding health profiles 152 are stored. The health profiles 152 contain health data that may include a variety of health and wellness characteristics about individuals. The health data may be that of users of the system, or health data relating to known wellness outcomes. Health and wellness characteristics may include various characteristics such as age, race, gender, height, weight, body fat percentage, or activity level.

In some embodiments, the health data may also include genetic information, such as particular genetic panels linked with certain conditions or diseases. Multiple genes collectively influence the likelihood of developing a variety of conditions or diseases. Knowledge of these gene combinations may be used to predict a person's risk for a particular disease. Such information may be obtained by genotyping, or obtaining an individual's DNA sequence by using biological assays.

An individual user health profile may be developed by obtaining user health data through any of the client devices 120A-120N, or through an outside provider 130. The outside provider may include any entity that possesses health or wellness information about an individual, such as a health care provider, physical trainer, or masseuse. For example, a user may provide current health information by filling out a simple survey inquiring into the user's demographic information, and standard health or wellness questions. The user's personal physician may update the information by providing historical health data, such as dates and outcomes of surgical procedures, or various test results. Additionally, genetic panel information may be provided through either the personal physician or other source. A number of private companies have made genome sequencing accessible to the public. In some embodiments, those private companies may provide a user's genetic sequencing information directly into the system 100. An individual's health profile may then be stored with the health profiles 152 of the server 150. The health profiles may continually be updated, as new health data may be provided or may change over time. Typically, genotypical data will remain static, but some phenotypical data such as weight, or blood pressure may change from time to time.

To maintain their wellness condition, or to achieve particular wellness goals, a user may provide those goals into the system 100. These goals may be include preventative goals (i.e. minimize the risk of hypertension or orthopedic injury), or aspirational goals (i.e. weight reduction, or pain management). A recommendation engine 156 may obtain the user health profile and user wellness goals from health profiles 152 to generate a user wellness recommendation. The recommendation engine 156 may do this by accessing a cohort module 154 that parses through the health profiles to create specific cohort groups based on one or more characteristics such as age, race, gender, height, weight, body fat percentage, activity level, or contents of a specific genetic panel. The recommendation engine 156 may use machine learning algorithms or fuzzy logic to best determine the appropriate cohort to compare or associate a particular user, with respect to a particular wellness goal. As shown in FIG. 1, the cohort module 154 may be a separate software module within the server, but in other embodiments the functionality of the cohort module 154 may be incorporated within the recommendation engine 156. The recommendation engine 156 may also access health intelligence data 158 which may include information provided by health care professionals relating to treatments or observations concerning various cohort groups. For example, health intelligence data may be knowledge based on clinical trials or medical publications relating to genetic markers that correlate with certain diseases or conditions.

In particular, an individual's genetic make-up may play a large role in how well they respond to a certain wellness recommendation. Therefore, knowing their genetic content can change aspects of the recommendation they receive. For example, if a user has a knee injury and is interested in pain management techniques to reduce joint inflammation and pain, the recommendation engine 156 may find that members of a cohort based on age, gender, activity level, and particular gene panel tend to respond well to certain pain management recommendations, and provide similar recommendations to the user. In embodiments of the invention, the user can then provide feedback as to the efficacy of the wellness recommendation back into the system to help further develop the cohort information for future user wellness recommendations.

Once this information has been generated, it can be provided to the user and/or their health professional through some interface, such as a display. The user may be continually monitored through any number of methods, including through personal health monitoring devices. As monitored user health data is obtained, the system can revise the user wellness recommendation based on the monitored user health data.

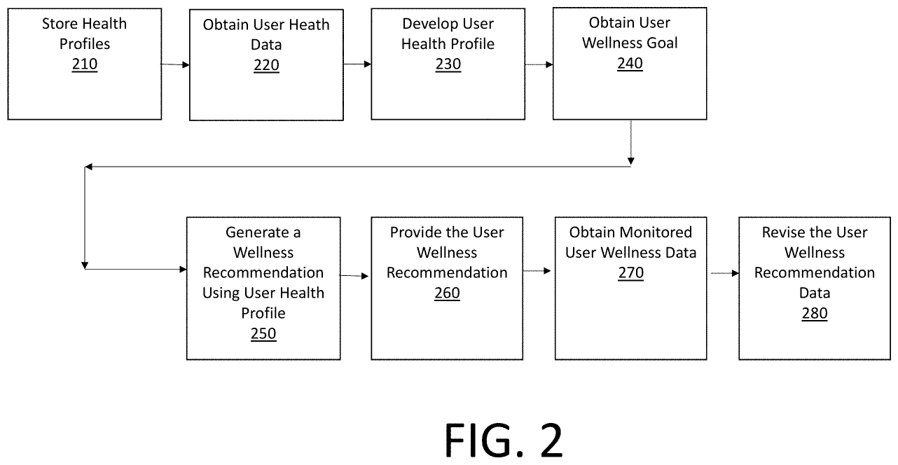

FIG. 2 is a flow diagram that demonstrates a method for providing interactive personal wellness recommendations consistent with to principles of the invention. As discussed above, the system stores health profiles at 210 within the system, wherein the health profiles include health data that may relate to a variety of different characteristics, including, but not limited to, physiological, health, wellness and genotypical information.

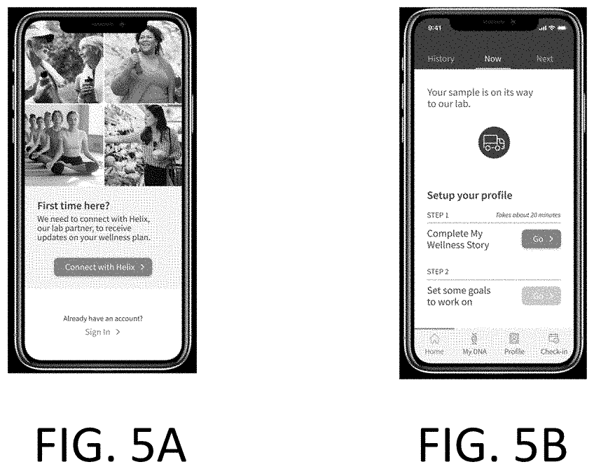

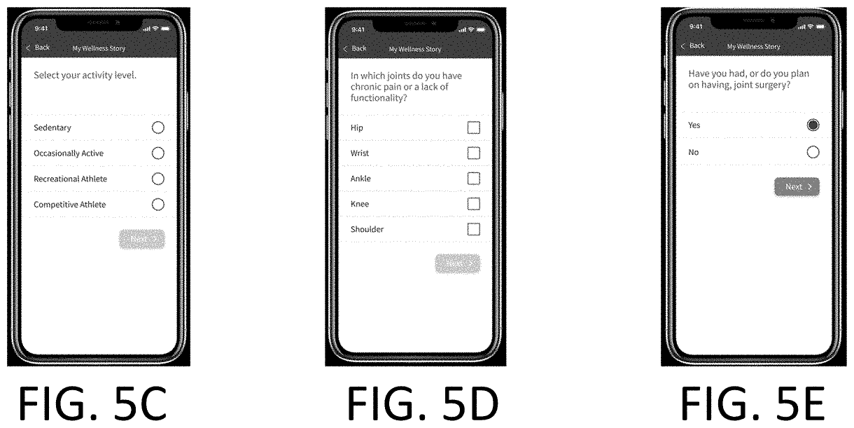

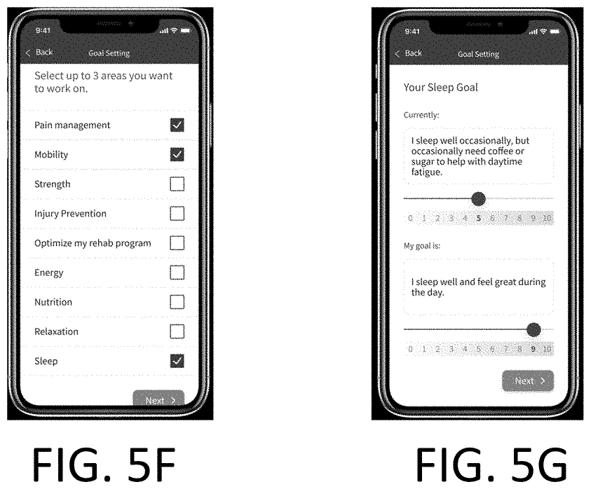

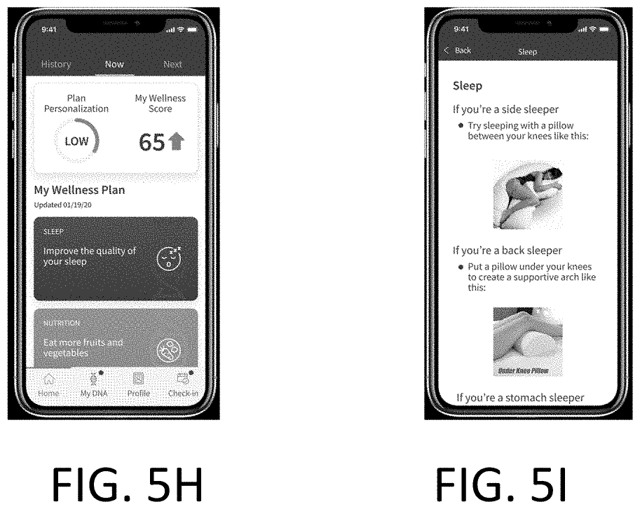

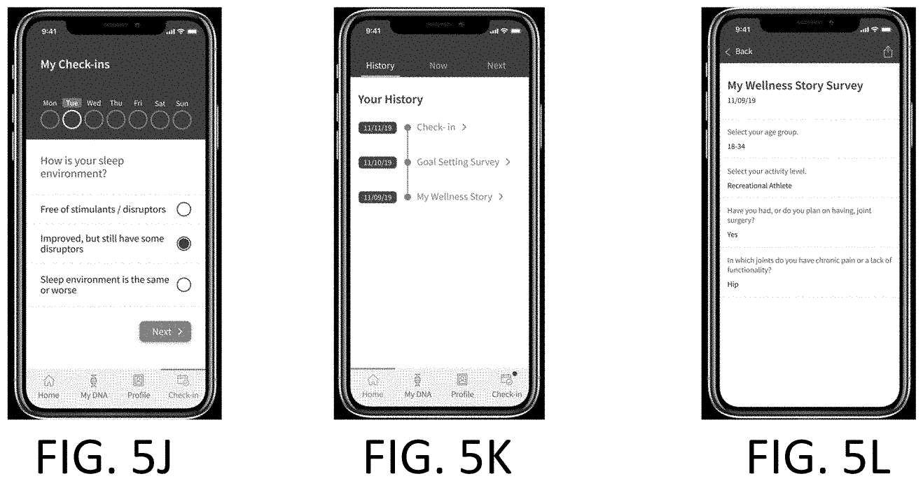

The system then obtains user health data at 220. As mentioned above with respect to FIG. 1, the data may be provided directly by the user, one or more service providers of the user or some combination of both. Typically, the information is entered into the system by the user or a provider through the use of an internet website portal, or through a mobile app. In some embodiments, as shown with respect to the interface of FIG. 5A, when a user accesses the system through a mobile app, the user may initiate the system by connecting the local application to a service provider, such as a genetic testing partner, to enable the provision of certain clinical information about the user from the provider. In other embodiments, multiple service providers may be connected into the system to contribute personalized information concerning the user. As shown in the interface of FIG. 5B, the user may use the interface to begin the process of providing user health data to the system.

FIGS. 5C-5E illustrate sample survey information presented to the user in sequential form to obtain user health data. As shown in FIGS. 5C-5E, the user is able to self-report characteristics such as activity level, current health status, and any personal health history. FIG. 5C asks the user to assess their activity level based on a four-tiered classification. FIG. 5D asks the user to identify any chronic joint pains. FIG. 5E asks the user about joint surgery history or plans. Referring back to FIG. 2, this user health data is used to develop a user health profile at 230. While the particular characteristics of FIGS. 5C-5E focus on physical activity and joint injury, the health profile may include questions relating to other health and wellness attributes of the user, and the system may ask any number of questions to develop a robust health profile.

A user provides one or more user wellness goals at 240. These wellness goals may be wellness goals directed at either achieving a certain target health characteristic, or may relate to maintaining a health characteristic at a certain state. In embodiments consistent with principles of the invention, the user wellness goal may be pain management for an orthopedic injury. The interfaces shown in connection with FIGS. 5F and 5G show an example goal setting inquiry. Initially, in FIG. 5F a user may select up to 3 health or wellness areas to address, such as pain management, mobility or sleep. The interface of FIG. 5G shows one example of a more specific goal setting under the area of “sleep.” As shown, the goal setting may be made relative to a scaled assessment of current sleep satisfaction, the scaled assessment clarified using plain language.

The user health profile and user health goal are accessed to generate a wellness recommendation at 250. As mentioned above, the recommendation may be based on an analysis of the efficacy of recommendations for similarly situated users based on membership in a variety of cohorts. Based on the user information, the system can determine an appropriate cohort based on one or more health characteristics to evaluate the potential efficacy of different wellness recommendations. Taking the example of the user health goal relating to pain management, the system may evaluate the user profile and compare the user to others in a similar age range, gender, activity level (e.g. engaged in casual exercise activity vs. marathon runner), and similar genetic predisposition to orthopedic injuries or healing capacities. In addition to the health data of the various cohort groups in the system health profiles, the system may also rely on health intelligence data that may be compiled from medical literature, or health professional observations.

The system then provides the user wellness recommendation to the user at 260. This recommendation may be provided through an e-mail, or through a secure sign-in web portal, or the mobile app. FIG. 5H shows a sample recommendation dashboard provided on a mobile app where user wellness goals may be organized and presented to the user. In the embodiment illustrated in connection with FIG. 5H, the dashboard provides the user with an assessment of personalization level of the plan, and a “wellness score” of the user. The personalization level may be based on how complete the user health data, given the amount and types of data that could be provided. The interface of FIG. 5I provides an example of a wellness recommendation provided relating to sleep improvement. The system then continues to monitor the user's progress with the user health goal at 270, which may be provided through periodic check-ins initiated by the system or independently by the user. FIG. 5J shows an interface of a sample check-in function on the mobile dashboard wherein the user can provide a day by day assessment of their sleep quality. Depending on the user health goal, the progress may also be monitored by a personal health monitoring device.

FIG. 5K shows the user history on the system, so the user can see their user health profile (labeled as “My Wellness Story”), their goals, and feedback into the system. The interface of FIG. 5L shows a summary of the user health profile, which can be updated by the user. Referring back to FIG. 2, the system then uses the monitored user wellness data to further develop the system health profiles to revise and improve the wellness data for making user wellness recommendations at 280. In embodiments consistent with principle of the invention, user feedback on goal progress and continued updates to the user health profile can continually be used to build better decision logic in a recommendation engine.

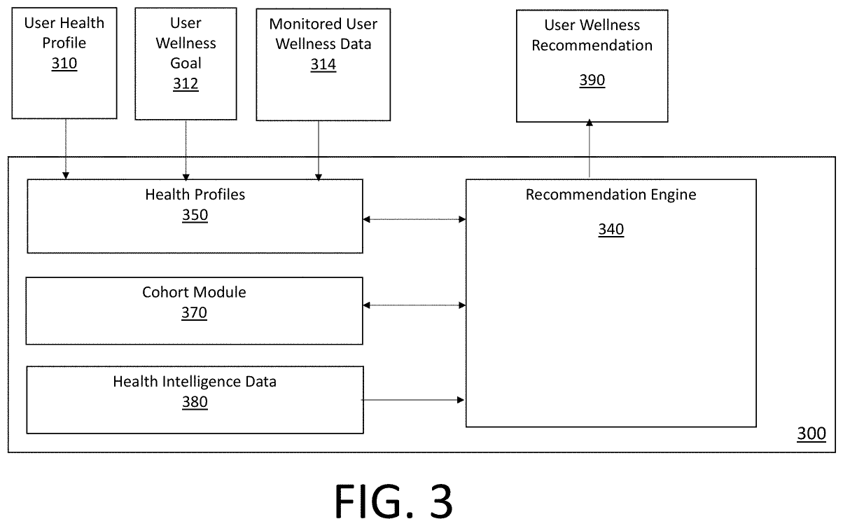

FIG. 3 illustrates an exemplary data flow according to some embodiments consistent with principles of the invention. The server 300 includes a memory 350 for storing health profiles. A user health profile 310 comprising user health data is sent to the server 300 where it is stored among the health profiles in the memory 350. In various embodiments, additional health data associated with the user can be provided in separate data flows to further develop the user health profile. For example, monitored user wellness data 314 can be provided back to the server 300 periodically to update the health profile stored in memory 350. The user provides a user wellness goal 312 associated with the user to the server 300. The recommendation engine 340 accesses the health profiles stored in memory 350 to analyze a user wellness goal 312 and associated user health profile. The recommendation engine 340 may be coupled to a cohort module 370. The recommendation engine 340 may call to the cohort module 370 to determine the optimal cohort of the user, given both the characteristics of user health profile and the particular user wellness goal. In addition, the recommendation engine 340 may access health intelligence data 380, which may include data relating to treatments or observations concerning various cohort groups. As mentioned above, health intelligence data may be knowledge based on clinical trials or medical publications relating to genetic markers that correlate with certain diseases or conditions. Using the available data and cohort determination, the recommendation engine 340 may provide a user wellness recommendation 390 to the user.

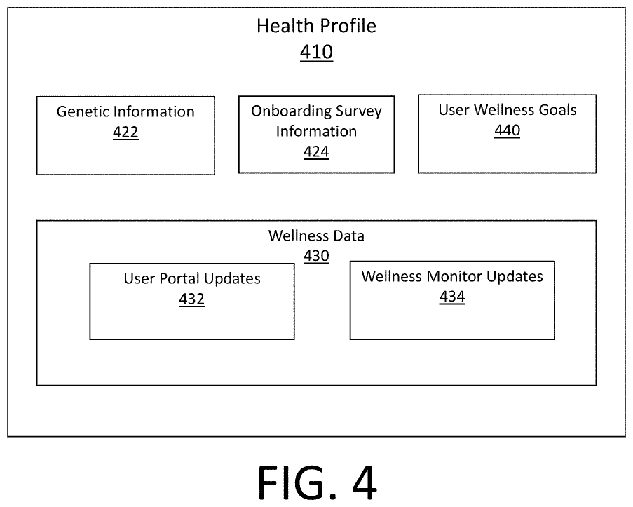

FIG. 4 is a representation of the data stored in connection with a user health profile 410 that may be used in systems according to principles of the present invention. The user health profile 410 may be comprised of health characteristics of the user. These characteristics may include genetic information 422, such as the results of a gene panel. In addition, the characteristics may include onboarding survey information 424 that may be submitted by the user or by a health professional. The user health profile may also include wellness data 430 that may be more dynamic in that the characteristics are likely to change with some frequency, and wherein maintaining historical changes would be beneficial for monitoring. This wellness data 430 may include data that is provided as updates 432 through a user portal. In embodiments, this may involve periodic questionnaires sent to the user that require a reply submission, or information provided by the user of their own accord. In addition, the wellness data 430 may include wellness monitor updates 434. The wellness monitor updates 434 may include wellness information supplied by a wearable health monitor, or monitored information supplied by a health provider, such as a physician, physical therapist, or other health professional.

Additionally, in some embodiments, the user wellness goals 440 may be stored as part of the health profile. In other embodiments consistent with principles of the invention, a user wellness goal 440 may be stored separately, but associated with the associated user health profile.

While example embodiments have been particularly shown and described, it will be understood by those skilled in the art that various changes in form and details may be made therein without departing from the scope encompassed by the appended claims.

It should be understood that the example embodiments described above may be implemented in many different ways. In some instances, the various methods and machines described herein may each be implemented by a physical, virtual or hybrid general purpose computer having a central processor, memory, disk or other mass storage, communication interface(s), input/output (I/O) device(s), and other peripherals. The general purpose computer is transformed into the machines that execute the methods described above, for example, by loading software instructions into a data processor, and then causing execution of the instructions to carry out the functions described, herein.

As is known in the art, such a computer may contain a system bus, where a bus is a set of hardware lines used for data transfer among the components of a computer or processing system. The bus or busses are essentially shared conduit(s) that connect different elements of the computer system, e.g., processor, disk storage, memory, input/output ports, network ports, etcetera, which enables the transfer of information between the elements. One or more central processor units are attached to the system bus and provide for the execution of computer instructions. Also attached to system bus are typically I/O device interfaces for connecting various input and output devices, e.g., keyboard, mouse, displays, printers, speakers, etcetera, to the computer. Network interface(s) allow the computer to connect to various other devices attached to a network. Memory provides volatile storage for computer software instructions and data used to implement an embodiment. Disk or other mass storage provides non-volatile storage for computer software instructions and data used to implement, for example, the various procedures described herein.

Embodiments may therefore typically be implemented in hardware, firmware, software, or any combination thereof.

In certain embodiments, the procedures, devices, and processes described herein constitute a computer program product, including a non-transitory computer-readable medium, e.g., a removable storage medium such as one or more DVD-ROM' s, CD-ROM's, diskettes, tapes, etcetera, that provides at least a portion of the software instructions for the system. Such a computer program product can be installed by any suitable software installation procedure, as is well known in the art. In another embodiment, at least a portion of the software instructions may also be downloaded over a cable, communication and/or wireless connection.

Further, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etcetera.

It also should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

While this invention has been particularly shown and described with references to example embodiments thereof, it will be understood by those skilled in the art that various changes in form and details may be made therein without departing from the scope of the invention encompassed by the appended claims.

What is claimed is:

1. A computer-implemented method, the method comprising:
storing in a computer memory information regarding health profiles, wherein health profiles include health data that is associated with at least one cohort group;
obtaining user health data about a user to develop a user health profile;
obtaining a user wellness goal;
assigning the user to at least one cohort group based on the user health profile;
generating a user wellness recommendation for obtaining the user wellness goal, at a recommendation engine, based on the membership in at least one cohort group of the user health profile by correlating the particular health profile against effective wellness regimens for the user wellness goal as it relates to the cohort group;
providing the user wellness recommendation;
obtaining monitored user wellness data;
analyzing user compliance with the wellness recommendation and the monitored user wellness data relative to the user wellness goal to determine user wellness progress; and
revising the user wellness recommendation based on the user wellness progress.

2. The method of claim 1 wherein health data includes user genotype data.

3. The method of claim 1 wherein health data includes user wellness data.

4. The method of claim 1 wherein monitored user wellness data includes data from a wearable health device.

5. The method of claim 1 wherein the user wellness goal includes pain management.

6. The method of claim 5 wherein the user health profile includes genetic markers.

7. The method of claim 6 wherein the user health profile includes genetic markers related to orthopedic conditions.

8. The method of claim 1 wherein the recommendation engine compares the user heath profile to health intelligence data to generate the user wellness recommendation.

9. The method of claim 1 wherein monitored user wellness data includes survey data provided by the user.

10. The method of claim 1 wherein the user wellness recommendation is provided through a device interface.

11. The method of claim 1 wherein the user wellness recommendation is provided through an e-mail sent to the user.

12. The method of claim 1 further comprising:
obtaining progress data relating to outcomes associated with compliance with the user wellness recommendation; and
revising health data associated with at least one cohort group to which the user is assigned.

13. The method of claim 1 further comprising:
accessing the user wellness recommendation and periodic monitored user wellness data, at a progress engine, to generate wellness tracking data; and
providing the wellness tracking data through a display device.

14. A computer-implemented method, the method comprising:
storing in a computer memory information regarding health profiles, wherein health profiles are associated with at least one cohort group
obtaining user health data to develop a user health profile;
assigning the user to at least one cohort group based on the user health profile;
obtaining a user wellness goal;
generating a user wellness recommendation for obtaining the user wellness goal based on the membership in at least one cohort group of the user health profile by correlating the particular health profile against effective wellness regimens for the user wellness goal as it relates to the cohort group;
providing the user wellness recommendation through a display;
periodically obtaining monitored user wellness data; and
analyzing user compliance with the wellness recommendation and the monitored user wellness data relative to the user wellness goal to determine user wellness progress; and
revising the user wellness recommendation based on the user wellness progress.

15. The method of claim 14 wherein health data includes user genotype data.

16. The method of claim 14 wherein health data includes user wellness data.

17. The method of claim 14 wherein monitored user wellness data includes data from a wearable health device.

18. The method of claim 14 wherein the user wellness goal includes pain management.

19. The method of claim 18 wherein the user health profile includes genetic markers.

20. The method of claim 19 wherein the user health profile includes genetic markers related to orthopedic conditions.

21. The method of claim 14 wherein the recommendation engine compares the user heath profile to health intelligence data to generate the user wellness recommendation.

22. The method of claim 14 wherein monitored user wellness data includes survey data provided by the user.

23. The method of claim 14 wherein the user wellness recommendation is provided through a device interface.

24. The method of claim 14 wherein the user wellness recommendation is provided through an e-mail sent to the user.

25. The method of claim 14 further comprising:
obtaining progress data relating to outcomes associated with compliance with the user wellness recommendation; and
revising health data associated with at least one cohort group to which the user is assigned.

26. The method of claim 14 further comprising:
accessing the user wellness recommendation and periodic monitored user wellness data, at a progress engine, to generate wellness tracking data; and
providing the wellness tracking data through a display device.

27. A computer-system for providing a wellness recommendation, the system comprising:
a data store in a computer memory, the data store storing information regarding health profiles, wherein health profiles are associated with at least one cohort group;
a user interface configured to:
i. receive user health data about a user to develop a user health profile,
ii. receive a user wellness goal; and
iii. monitor user wellness data;
one of more processors coupled to the user interface and configured to:
i. access the data store;
ii. assign the user to at least one cohort group based on the user health profile;
iii. generate a user wellness recommendation for obtaining the user wellness goal based on the membership in at least one cohort group of the user health profile by correlating the particular health profile against effective wellness regimens for the user wellness goal for the cohort group;
iv. provide the user wellness recommendation through the user interface;
v. analyze user compliance with the wellness recommendation and the monitored user wellness data relative to the user wellness goal to determine user health progress
vi. revise the user wellness recommendation based on the user wellness progress.

28. The system of claim 27 wherein health data includes user genotype data.

29. The system of claim 27 wherein health data includes user wellness data.

30. The system of claim 27 wherein monitored user wellness data includes data from a wearable health device.

31. The system of claim 27 wherein the user wellness goal includes pain management.

32. The system of claim 31 wherein the user health profile includes genetic markers.

33. The system of claim 32 wherein the user health profile includes genetic markers related to orthopedic conditions.

34. The system of claim 27 wherein the recommendation engine compares the user heath profile to health intelligence data to generate the user wellness recommendation.

35. The system of claim 27 wherein monitored user wellness data includes survey data provided by the user.

36. The system of claim 27 wherein the user wellness recommendation is provided through a device interface.

37. The system of claim 27 wherein the user wellness recommendation is provided through an e-mail sent to the user.

38. The system of claim 27 further comprising:
obtaining progress data relating to outcomes associated with compliance with the user wellness recommendation; and
revising health data associated with at least one cohort group to which the user is assigned.

39. The method of claim 27 further comprising:
accessing the user wellness recommendation and periodic monitored user wellness data, at a progress engine, to generate wellness tracking data; and
providing the wellness tracking data through a display device.
